(19) United States
(12) Patent Application Publication
(10) Pub. No.: US 2023/0321444 A1
(43) Pub. Date: Oct. 12, 2023

(54) AUTOMATIC TITRATION FOR VAGUS NERVE STIMULATION

(71) Applicant: The Alfred E. Mann Foundation for Scientific Research, Valencia, CA (US)

(72) Inventors: Brian Michael Shelton, Altadena, CA (US); Brian Mech, Buffalo, MN (US); Valma Klein, Santa Clarita, CA (US); Neil Talbot, La Crescenta, CA (US); Hitesh Hotlani, Santa Clarita, CA (US)

(21) Appl. No.: 18/193,386

(22) Filed: Mar. 30, 2023

Related U.S. Application Data
(60) Provisional application No. 63/325,566, filed on Mar. 30, 2022.

(51) Int. Cl. A61N 1/36; A61N 1/05; A61B 5/00

(52) U.S. Cl. CPC A61N 1/36139; A61B 5/4848; A61N 1/0556

(57) ABSTRACT

The disclosure provides systems and methods for automatically titrating an electrical pulse amplitude for a patient-implanted VNS stimulator. One or more external sensors (e.g., EEG, EKG, EMG, auditory sensors, inertial motion sensors, etc.) can be applied to the patient to generate data relevant to an acceptable amplitude of the electrical pulse for a given cathode in a multi-cathode cuff. In one embodiment, the device may include a controller on the implanted VNS stimulator that receives data, e.g., using a wireless connection, from the external sensors and titrates upward the amplitude until an acceptable amplitude is determine that provides efficacy with minimal, if any, side effects.

### CROSS-REFERENCE TO RELATED APPLICATION

This application claims the benefit of U.S. Provisional Application No. 63/325,566, entitled “AUTOMATIC TITRATION FOR VAGUS NERVE STIMULATION,” which was filed on Mar. 30, 2022, the entire contents of which is expressly incorporated by reference herein in its entirety.

### BACKGROUND

Epilepsy and depression are two extremely common maladies. Epilepsy produces potentially-fatal seizures. Both conditions can be treated under appropriate circumstances with vagus nerve stimulation (VNS). VNS entails the surgical implantation of a stimulator device into a patient’s chest area under the skin to stimulate the vagus nerve with electrical stimulus pulses. The vagus nerve originates from the brainstem and traverses both sides of the neck down to the chest and abdomen. The VNS device sends electrical signals via the vagus nerve to the brain. A lead wire having a cuff at the proximal end connects the stimulator device to the vagus nerve. The cuff has one or more electrodes within the cuff and, when implanted, encircles the vagus nerve. VNS has been shown to be helpful in many cases for reducing the number and severity of seizures, particularly for patients who are less responsive to more non-invasive methods like oral medication. VNS has also been shown to reduce depression in certain treatment-resistant patients.

For conventional VNS devices, a clinician or other professional (collectively herein “clinician”) sets a default pulse width and frequency for the electrical stimulus pulses to be periodically transmitted. Starting at a low stimulus amplitude, the clinician increases the amplitude or value of the stimulation current associated with the pulses to an amplitude or value that is efficacious for treating epilepsy or depression. The increase is conventionally performed slowly in step sizes, for example, on a biweekly basis, until the patient begins to experience side effects such as dry mouth or hoarseness. In typical systems, the clinician may increase the stimulation or stimulus current amplitude (also referred herein as “amplitude” or “pulse amplitude”) by a fixed amount, such as 0.25 milliamps (“mA”) every other week until she or he observes side effects. It may also be the case that the clinician may need to reduce the stimulus current amplitude during the weeks or months of post- implant healing to find and set the optimal stimulus amplitude in order to achieve efficacy, while minimizing side effects.

Often there is some instability in tissue impedance within and outside the cuff during the weeks and several months of healing which affects nerve stimulation thresholds and optimal stimulus amplitudes. In treating epilepsy and seizures, one significant problem with the existing approach is that it starts and proceeds slowly. The patient remains vulnerable to seizures during the weeks that the clinician attempts to fix on an optimal stimulation pulse amplitude. Although the titration procedures can be automated, the clinician needs to get involved when the patient experiences side effects. The clinician may often rely on the subjective experiences of the patient’s description of side effects when deciding whether to change the stimulus amplitude and to achieve the optimal amplitude. In short, achieving a balance between a maximum efficacy of the VNS stimulator with a minimum pulse amplitude to achieve the benefits of the procedure without causing pronounced side effects or introducing other detrimental health deficiencies is time consuming and prone to human error. Because of the slowness of completing titration, it can take 6 to 12 months before the patient experiences a noticeable change in their seizure rate or seizure duration. The present disclosure addresses these and other shortcomings in the art.

### BRIEF SUMMARY OF EXEMPLARY ASPECTS OF THE DISCLOSURE

For the purposes of this disclosure “titration” means the process of finding the optimal stimulus parameters, e.g., stimulus pulse amplitude, stimulus pulse width, and stimulus frequency. To titrate also means to find the optimal stimulus parameters. When pulse width and frequency are pre-set, then titration requires finding the optimal stimulus amplitude. “Titration” may also include initially choosing which electrodes are used for delivering stimulation within an array of electrodes, for example, among multiple electrodes in a cuff. Some examples include choosing a single cathode electrode within an array of electrodes (monopolar or unipolar stimulation), or a combination of cathode electrodes within an array of electrodes or a combination of two or more electrodes in an electrode array, with at least one electrode operating as cathode and at least one electrode operating as anode (bipolar stimulation).

Applicants have recognized that if clinicians knew what pulse amplitude values caused side effects in a patient, the VNS stimulation titration can commence at a higher starting pulse amplitude, thereby shortening the time between the start of therapy and a reduction in seizure frequency. An acceptable amplitude may include an identified amplitude (often but not necessarily a minimum effective amplitude) that is effective in reducing seizures without introducing pronounced side effects in the patient or inducing other physical events that may contribute to health problems. An acceptable amplitude includes both a determined amplitude at the beginning of the process, as well as changed amplitudes as a consequence of future amplitude adjustments that may occur over even or uneven intervals on a periodic basis. Where an EMG or EKG are involved, an acceptable amplitude may also include an amplitude where side effects are just becoming observable. In various embodiments, the amplitude may be set at this point, or reduced if side effects are too pronounced, or to titrate downward until the side effects disappear. Such a modified amplitude is considered an acceptable amplitude for purposes of this disclosure. Furthermore, where multiple electrodes are involved, an acceptable amplitude may be achieved by titrating selected electrodes. An acceptable or optimal amplitude may be different for different selected electrodes functioning as cathodes within an array of electrodes.

In addition to identifying a higher starting amplitude, Applicants have recognized that, where physiologic signals relating to detected events in the patient can be monitored regularly, the rate of the upward titration process can be potentially dramatically increased, provided that there are no unwanted side effects.

Aspects of the present disclosure consequently include using temporary and detachable external sensors to titrate the stimulation pulse amplitude. One or more external sensors as described herein can be used to detect physical events in the patient that, in turn, can be used to inform the clinician of afferent or efferent stimulation of the vagus nerve. In some embodiments, one or more electroencephalogram (EEG), electrocardiogram (EKG) and/or electromyogram (EMG) sensors can be used to titrate the pulse amplitude and select active electrodes in an array of electrodes after implantation of the VNS stimulator and the surgical recovery period.

In other embodiments, the titration process may be automated to varying degrees as disclosed herein. The VNS stimulator may include a controller configured to receive electronic signals (wireless or otherwise) from one or more external sensors equipped with Low Energy Bluetooth or another technology, and/or via input from an external processing system, for example data representing measurements from an EMG. The received information from the external sensors include information that can be used by the controller to automatically titrate the stimulation pulses to an acceptable level. In other embodiments, the information from the external sensors can be used by the clinician to titrate the pulses. For example, the clinician, or a processing system available to the clinician, can send instructions to the controller based at least in part on the sensor output to change the amplitude of the pulse while observing the patient for side effects. In other embodiments, information from internal and external sensors can be used by the clinician to not only titrate the pulses but also to select different electrodes of optimum titration by sending the changes to controller to be programmed remotely into the implantable.

As a result of the physical events detected by these external sensors, the concepts herein allow for a fast titration of the VNS pulse amplitude. These benefits extend, for example, to a fast determination of a maximum pulse amplitude (using the information from the external sensor(s) and/or the side effects identified in or reported by the patient) on each electrode of a multi-electrode stimulation cuff that may be affixed to the vagus nerve. With these sensors, the disclosure enables an automatic determination of stimulation efficacy by monitoring for seizures - e.g., monitoring an EKG sensor for ictal tachycardia events or monitoring an EEG sensor for seizure activity events.

Aspects herein further permit automatic titration of the stimulation amplitude to the point where seizure occurrence is reduced with minimal stimulator current consumption. Among other benefits, these factors can allow for increased VNS stimulator longevity and longer recharge intervals for rechargeable systems. Active electrodes, as opposed to unused electrodes, can also be identified in a multi-electrode stimulation cuff that achieve the best therapeutic outcome.

Accordingly, in one aspect of the disclosure, a system for vagus nerve stimulation (VNS) comprises a VNS stimulator implanted in a patient and configured to transmit periodic or episodic electrical stimulation pulses to a vagus nerve of the patient; and a sensor configured to detect from the patient a biological signal, wherein the VNS stimulator comprises a controller configured to automatically titrate at least one of a VNS pulse stimulus parameter, among stimulus pulsewidth, stimulus amplitude, stimulus frequency and duty cycle, based at least in part on the biological signal until an acceptable stimulation result is achieved.

In some aspects, the sensor comprises an electroencephalogram (EEG) sensor, the EEG sensor being positioned on a patient’s scalp or behind a patient’s ear. In some aspects, the sensor comprises an electrocardiogram (EKG) sensor. In some aspects, the sensor comprises an electromyography (EMG) sensor. In some aspects, the sensor comprises a microphone, and the biological signal comprises a heart rate or an ictal tachycardia event. In some aspects, the sensor comprises a plurality of sensors that detect muscle or neural electrical signals.

In some aspects, the biological signal comprises a predetermined electrical activity in a patient’s brain. In some aspects, the biological signal comprises one or both of an intensity or frequency of ictal tachycardia or bradycardia events.

In some aspects, the EMG sensor is configured to be positioned on or proximate to a larynx of the patient and to detect, as the event, stimulation information of the laryngeal branch of the vagus nerve; and the controller is configured to titrate up at least one of the VNS pulse stimulus parameters, which includes pulse width, pulse frequency, pulse amplitude, frequency and duty cyle, over a period of time based on stimulation information including side effects of the patient relating to a detected stimulation of the laryngeal nerve.

In some aspects, the controller is configured to automatedly use the biological signal to determine a unique stimulation threshold for at least one electrode of the VNS stimulator when the VNS stimulator is configured using either a single electrode, or configured using a multi-electrode stimulation cuff or lead. In some aspects, the controller is configured to determine a VNS stimulation threshold by increasing the VNS pulse amplitude at one or more intervals over time until the sensor detects a biological signal from the patient relevant to an acceptable amplitude of the stimulation pulses.

In some aspects, the controller is configured to determine an efficacy of the VNS stimulator based on measurements from an EKG sensor or an EEG sensor, wherein the measurements from the EKG sensor include any one or more of heart rate variability (HRV) measurements, bradycardia events, or tachycardia and fibrillation events.

In some aspects, the measurements from the EKG sensor comprise one or more of a number of ictal tachycardia events, a number of bradycardia events, or a Heart Rate Variability (HRV); or the measurements from the EEG sensor comprise indications of a seizure event.

In some aspects, the controller is configured to automatically effect an optimal titration of the VNS stimulation amplitude such that seizure events are reduced with minimal stimulator use as determined based at least in part on the event.

In some aspects, the VNS stimulator includes a conductor coupled between the VNS stimulator and one or more multi-electrode stimulation cuffs attached to the vagus nerve.

In some aspects, the controller is configured to determine an optimal stimulation amplitude for each cathode of the one or more multi-electrode stimulation cuffs by monitoring a number of one or both of ictal tachycardia events or bradycardia events during successive time periods when each cathode or cathode pair of the one or more multi-cathode stimulation cuffs is activated with the stimulation pulses.

In another aspect of the disclosure, a method for vagus nerve stimulation (VNS) comprises transmitting periodic electrical stimulation pulses from a VNS stimulator implanted in a patient to a vagus nerve; receiving, from a sensor external to the patient, data comprising a physical biological signal from the patient and relevant to an acceptable stimulation of the vagus nerve; and titrating an amplitude of the pulses upward based at least in part on the data. In some aspects, the received data is included in a wireless signal. It is understood that methods for VNS according to the disclosure may utilize any of the VNS systems or devices disclosed herein.

In still another aspect of the disclosure, provided herein are devices for automatically titrating a vagus nerve stimulation (VNS).

In some aspects, a device for automatically titrating VNS may comprise a VNS stimulator implanted in a patient and configured to transmit electrical stimulation pulses to a vagus nerve of the patient, the VNS stimulator comprising a controller configured to: receive data from an external sensor configured to detect from the patient a biological signal relevant to an acceptable amplitude of the stimulation pulses; and titrate an amplitude of the stimulation pulses based in part on the received data.

In some aspects, the sensor comprises an electroencephalogram (EEG) sensor, the EEG sensor being positioned on a patient’s scalp or behind a patient’s ear. In some aspects, the sensor comprises an electrocardiogram (EKG) sensor. In some aspects, the sensor comprises an electromyography (EMG) sensor. In some aspects, the sensor comprises a microphone, and the event comprises a heart rate or an ictal tachycardia event. In some aspects, the sensor comprises a plurality of sensors that detect muscle or neural electrical signals.

In some aspects, the event comprises electrical activity in a patient’s brain. In some aspects, the event comprises one or both of an intensity or frequency of ictal tachycardia or bradycardia events.

In some aspects, the EMG sensor is configured to be positioned on or proximate to a larynx of the patient and to detect, as the event, efferent stimulation information of the vagus nerve; and the controller is configured to titrate at least one of the stimulus parameters among pulse amplitude, pulse width, pulse frequency and duty cycle, over a period of time based on stimulation information including side effects of the patient relating to a detected stimulation of the laryngeal nerve.

In some aspects, the controller is configured to automatedly use the stimulation information to determine a unique stimulation threshold for at least one electrode of the VNS stimulator when the VNS stimulator is configured using either a single electrode, or configured using a multi-electrode stimulation cuff or lead.

In some aspects, the controller is configured to determine a VNS stimulation threshold by increasing the amplitude at one or more intervals over time until the sensor detects a signal generated on the vagus nerve.

In some aspects, the controller is configured to determine an efficacy of the VNS stimulator based on measurements from an EKG sensor or an EEG sensor. In some aspects, the measurements from the EKG sensor comprise one or more of a number or intensity of ictal tachycardia events, a number of bradycardia events, or a Heart Rate Variability (HRV); or the measurements from the EEG sensor comprise indications of a seizure event.

In some aspects, the controller is configured to automatedly use the stimulation information to determine a unique stimulation threshold for at least one electrode of the VNS stimulator when the VNS stimulator is configured using either a single electrode, or configured using a multi-electrode stimulation cuff or lead.

In some aspects, the controller is configured to determine a VNS stimulation threshold by increasing the amplitude at one or more intervals over time until the sensor detects a signal generated on the vagus nerve.

In some aspects, the controller is configured to determine an efficacy of the VNS stimulator based on measurements from an EKG sensor or an EEG sensor.

In some aspects, the measurements from the EKG sensor comprise one or more of a number or intensity of ictal tachycardia events, a number of bradycardia events, or a Heart Rate Variability (HRV); or the measurements from the EEG sensor comprise indications of a seizure event.

In some aspects, the acceptable amplitude is such that seizure events are reduced with minimal stimulator use as determined based at least in part on the event. In some aspects, the acceptable amplitude comprises a stimulus amplitude that reduces severity of a patient’s seizures while minimizing or eliminating side effects.

In some aspects, the VNS stimulator includes a conductor coupled between the VNS stimulator and one or more multi-cathode stimulation cuffs attached to the vagus nerve.

In some aspects, the controller is configured to determine an optimal stimulation amplitude for each cathode of the one or more multi-cathode stimulation cuffs by monitoring a number of one or both of ictal tachycardia events or bradycardia events during successive time periods when each cathode or cathode pair of the one or more multi-cathode stimulation cuffs is activated with the stimulation pulses.

In some aspects, the controller is further configured to receive data comprising events detected by one or more external or internal sensors and transmit the received data to a clinician for remote monitoring. In some aspects, the controller is further configured to receive data, programmed remotely and comprising an instruction to titrate the VNS pulse parameters or select a electrode or electrodes that deliver stimulation.

In some aspects, a device for automatically titrating VNS may comprise a VNS stimulator implanted in a patient and configured to transmit electrical stimulation pulses to a vagus nerve of the patient, the VNS stimulator comprising a controller configured to: receive data from an external sensor configured to detect from the patient a physical event relevant to one or more stimulus parameters of the stimulation pulses; and titrate at least one or more stimulus parameters of the stimulation pulses to find one or more optimal parameters based in part on the received data.

In some aspects, a device for automatically titrating VNS may comprise a VNS stimulator implanted in a patient and configured to transmit electrical stimulation pulses to a vagus nerve of the patient; a cuff arranged on the vagus nerve and coupled to the VNS stimulator via a lead wire; electrodes coupled to the cuff, the electrodes contacting the vagus nerve at different positions; and a controller coupled to the VNS stimulator and configured to: receive data from an external sensor configured to detect from the patient a biological signal relevant to one or more acceptable stimulus parameters of the stimulation pulses from at least one of the electrodes; titrate one or more optimal parameters using different electrodes to find one or more optimal parameters and one or more optimal electrodes to activate as stimulating electrodes based in part on the received data.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

FIG. 1 is a diagram illustrating an exemplary embodiment of a VNS system for treating epilepsy using an implantable VNS stimulator coupled via a lead wire to a cuff electrode on the vagus nerve.

FIG. 2 is a diagram illustrating an exemplary embodiment of a VNS stimulator including a pulse generator and a controller configured to titrate one or more stimulation parameters, i.e., stimulus pulse width, stimulus frequency and stimulus amplitude .

FIG. 3 is a diagram illustrating an exemplary embodiment of a wire and cuff electrode for use in VNS stimulation.

FIG. 4 is a diagram of an exemplary EMG sensor unit and processing system used by a clinician in some embodiments.

FIG. 5 shows an example of a wireless EMG sensor system used by a clinician in some embodiments.

FIG. 6 shows another example of the wireless EMG sensor system for automatically sending data directly to the VNS stimulator in some embodiments.

FIG. 7 is an illustration of embodiment of example titration processes using an EMG sensor to detect physical events from the patient.

FIG. 8 is an example diagram of an electrocardiogram (EKG) sensor including electrodes attached to a patient and a device for interfacing with a processing system.

FIG. 9 is an example block diagram of a patient outfitted with different external sensors and an example sensor unit including a processing system according to an embodiment.

FIG. 10 is a conceptual diagram of an EEG sensor affixed to a patient’s head and a device for interfacing with a processing system.

FIG. 11 is a conceptual flow diagram of a process for performing automated VNS titration according to an embodiment.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Several aspects of exemplary embodiments according to the present disclosure will now be presented with reference to various systems and methods. These systems and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” or “controller” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), application-specific integrated circuits (ASICs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more exemplary embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random- access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

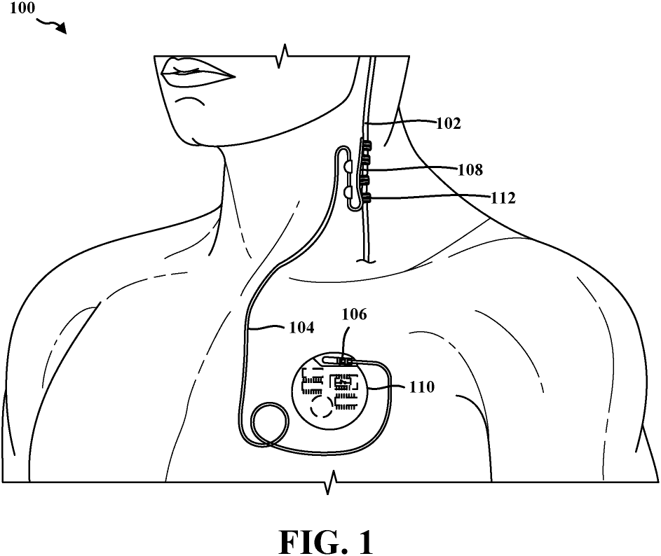

FIG. 1 is a diagram 100 illustrating an exemplary embodiment of a VNS system for treating epilepsy using a VNS stimulator 110 implanted under the skin in the chest of a patient and coupled via a lead wire 104 to a cuff electrode 108 on the vagus nerve 102. In the example shown, four electrode pairs 112 (for a total of 8 electrodes) are used to contact the vagus nerve. The electrodes may be selectively activated, e.g., to identify acceptable amplitudes for the different cathodes. Not all electrodes may be active-some electrodes may remain inactive, meaning they do not function as either cathodes or anodes. In some embodiments, a single electrode or multiple electrodes (all functioning as cathodes) are used and active and the metal housing portion of stimulator 110 may be used as a return electrode -- also known as an indifferent electrode or an anode. In other embodiments, pairs of electrodes (at least one cathode and one anode) are used in a bipolar electrode configuration. The stimulation of the vagus nerve using electrical pulses is believed to stabilize abnormal electrical activity in the brain which can lead to seizures. VNS stimulator 110 may also include a rechargeable battery which may be recharged inductively through the stimulator housing and through intact patient skin, i.e, transcutaneously. In some embodiments, the stimulator may include a single-use, primary-cell battery. VNS stimulator 110 may further include an outlet portion for enabling a connection between VNS stimulator 110 and lead wire 104, which enables current flow to the vagus nerve 102 via the cuff 108 and electrode(s) 112.

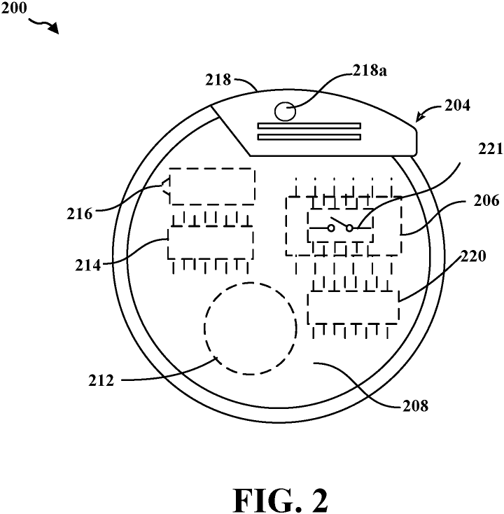

It should be expressly understood that the embodiment shown in FIG. 1 is a nonlimiting example provided solely for illustrative purposes. In other embodiments, alternative configurations may be used (e.g., a cuff geometry wherein some or all of the electrodes are enclosed within the cuff). For example, in some embodiments the cuff geometry of a VNS system according to the disclosure may use a configuration as shown in FIG. 2 of U.S. Pat. No. 10,967,178, or as shown in U.S. Pat. No. 4,602,624.

FIG. 2 is a diagram 200 illustrating an exemplary embodiment of a VNS stimulator 204 including a controller 220 with processing circuitry configured to titrate stimulus electrical pulse parameters, including the amplitude of the stimulation pulses, as described in greater detail herein. The VNS stimulator 204 may include a rechargeable battery 212 that can be accessible for recharging inductively. In some embodiments the battery 212 may be a single-use, primary cell battery. The battery 212 of VNS stimulator 204 may be configured to supply power to a pulse generator 206, which is programmed to generate the periodic electrical pulse having a set frequency and pulse width. The pulse generator can be activated and deactivated (the latter causing the stimulation pulses to the electrode or electrodes to be turned off) via a switch 221. The connections on the integrated circuits may be coupled together selectively via a small printed circuit board 208. In other embodiments, the VNS stimulator 204 may be implemented as an SoC on a die, or a packaged die.

VNS stimulator 204 further may include a transceiver/receiver 216. In some embodiments, transceiver 216 includes a wireless receiver configured to receive wireless signals, e.g., Bluetooth Low Energy, from a source external to the patient. In some embodiments, transceiver may further include a wireless transmitter, e.g., for providing feedback to a processor used in a clinician programmer device, or to an external sensor. In still other embodiments, the transceiver 216 may include a wire for receiving information from the vagus nerve or another part of the body. The wire may also extend outside the body for temporary connection to an external sensor or other processing. The wireless receiver in transceiver 216 may further be configured in some embodiments to receive instructions for the controller 220 to titrate the pulse. The wireless transceiver 216 may further be configured to receive information including events recorded or detected by one or more external sensors. When acting as a transmitter, the transceiver 216 can provide feedback signals to external sources using data generated by controller 220.

In some embodiments, the information received from the wireless receiver/transceiver 216 may be provided to a memory 214. The controller 220 may access the memory 214 to receive and process instructions to titrate upward or downward the generated electrical pulses, or to temporarily deactivate the pulse generator 206. In various embodiments, the controller 220 may access information including physical events detected by one or more external sensors (e.g., EMGs, EEGs, EKGs, microphones, etc.). The processor may evaluate the detected events and, in these embodiments, titrate the amplitude upward (or downward) as necessary. The pulse generator 206 may generate the electrical stimulus pulses accordingly, which may be provided to an outlet portion 218 to which the lead wire (see FIG. 1) is attached. In one embodiment, the outlet portion may include an aperture 218a for a jack to be inserted to attach the lead wire

While various functions of the VNS stimulator 204 have been shown, it will be appreciated by those skilled in the art upon review of this disclosure that different architectures may be used. For example, the controller 220 may include more than one integrated circuit, or it may include a separate module coupled to the VNS stimulator. The controller 220 may further include one or more general purpose processors, RISC processors, or other types of processors. The controller 220 may in some embodiments include dedicated hardware. For example, the controller 220 may be any one or more of a digital signal processor (DSP), a field programmable gate array (FPGA), an application- specific integrated circuit (ASIC), or a combination of digital logic devices.

The memory 214 may include any suitable memory, such as a combination of volatile and non-volatile memory, dynamic random access memory (DRAM), static random access memory (SRAM), read only memory, flash or other solid state memory, or the like. Other types of memory are possible. The battery 212 may supply the memory 214 with power as necessary. Non-volatile memory within memory 214 may be used to store critical settings to enable system reset, for example. Pulse frequency and pulse width values may be stored in the memory. In some embodiments, the memory may be accessible and programmable as noted above via the controller 220, or via data or instructions received at via wireless receiver 216. The memory 214 may also include firmware for use by the controller, or other program information for automatically titrating one or more of stimulus pulse width, pulse frequency and pulse amplitude.

The titration may be based, in part or in whole, on information received at the receiver 216 such as detected events (which are deemed to include detected patient characteristics and features using one or more external sensors) and other such information relevant to an acceptable stimulus amplitude, as further described herein. For purposes of this disclosure, information including events detected by external sensors may be considered relevant to an acceptable stimulus amplitude when the information is useful for enabling the controller 220 to determine whether to titrate the amplitude upward or downward (or to suspend titration activity), such that over time, an acceptable or generally optimal amplitude is identified for the patient.

One example of such an acceptable stimulus amplitude is an amplitude of the pulse generator that is just high enough to be clinically effective in reducing seizure activity but without producing significant or potentially dangerous side effects, if any. It will be appreciated that in other applications, the acceptable amplitude may be defined slightly differently. For example, a clinician may observe that while a lower amplitude is effective in reducing seizures, a slightly higher amplitude may be more effective and may reduce seizures, without the slightly higher amplitude producing unwanted side effects (or minimal side effects).

In short, the “acceptable” (e.g., optimal) amplitude of the electrical stimulus pulses may be slightly different, depending on the patient and the observed outcomes, for example. In some embodiments, an acceptable amplitude may fall within a range of amplitudes. Generally, smaller stimulus amplitudes may advantageously increase the time needed between recharges of the battery 212 in the VNS stimulator 204. Other considerations may dictate the need for different amplitudes (e.g., substantially increased efficacy in reducing seizures, or improving depression in cases where depression is indicated). The welfare of the patient remains a priority, and it is often beneficial to have a lower amplitude, as a lower current may be deemed less invasive in many cases. Shorter pulse widths and lower stimulus frequencies may have more or less side effects for a given stimulus amplitude.

It will be appreciated that VNS stimulator 204 need not be circular or elliptical in nature, and may take on different shapes based on different design considerations and patient needs. More generally, the components identified in the various figures may take on different geometries than those shown.

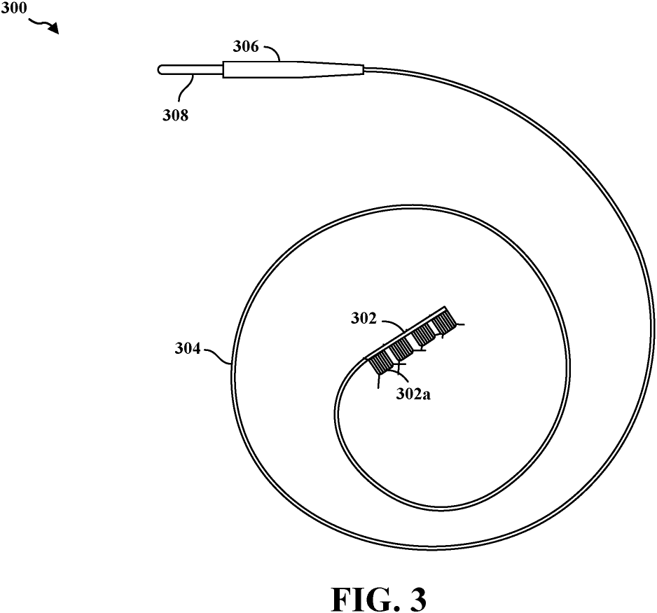

FIG. 3 is a diagram illustrating an exemplary embodiment of a wire and electrode cuff for use in VNS stimulation. The jack 308 may be inserted in one configuration into the aperture 218a (FIG. 2) of VNS stimulator 204. It will be appreciated that the components are not drawn to scale, and typically the jack 308 in this embodiment would be sized very small, and/or angled differently, to be minimally intrusive to the patient in which the component is implanted. In some embodiments, an insulating sheath 306 may provide support for the jack 308 as the insulated wire 304 terminates at the proximal end of a cuff electrode lead 302, which further illustrates a plurality of electrodes 302a or electrode pairs.

The vagus nerve is connected to various features in the voice box, as well as the heart, stomach, and other organs or parts of the human anatomy. For this reason it is not surprising that an excessive current amplitude may cause unwanted side effects, the milder of which may include hoarseness and dry mouth, along with a host of other possible symptoms. The symptoms may involve the throat, brain (e.g., headaches), and stomach (e.g., indigestion, stomach pain). The VNS implantation procedure is typically performed by a neurosurgeon. Stimulation titration may begin as early as during the implant period by the surgical staff, or following some recovery period.

The concepts disclosed herein may be used following the initial implantation procedure. In addition, even after use of the VNS stimulator commences, an assessment is performed periodically to re-assess the effectiveness of the VNS therapy. Particularly where a sub-optimal or less than expected efficacy is observed, the principles of this disclosure can be applied after each periodic assessment, for example, in order to reestablish the acceptable stimulus parameters, including amplitude of the stimulus pulse or to identify more acceptable stimulus parameters, including amplitude with the potential prospects of yielding greater results.

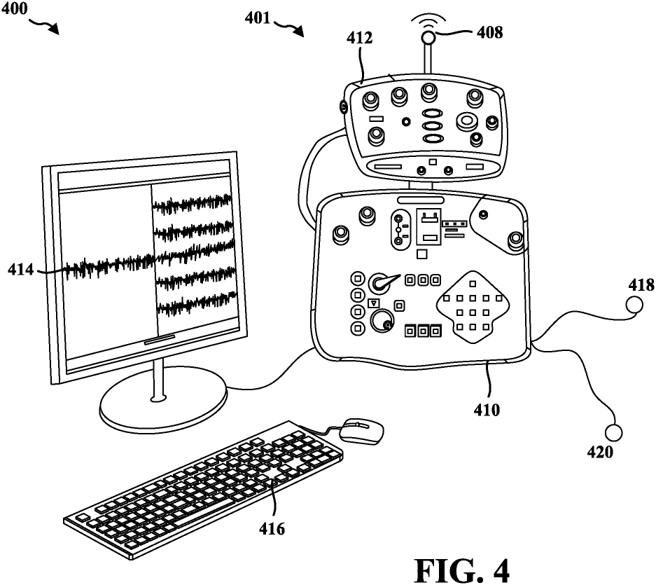

FIG. 4 is a diagram 400 of an exemplary EMG unit 401 used by a clinician in some embodiments. The EMG unit 401 may include a body 410 in which a processing system can be housed, and a controller 412 for tuning and manipulating the various options used in EMG measurement procedures. In some embodiments, the EMG may include wires for use with the patient including one or more electrodes 418, 420. As described below, in other embodiments, the EMG electrodes may be wireless, or they may be coupled to a Bluetooth or other wireless device. Information about events detected during EMG monitoring can be received by the EMG unit via antenna 408 in those embodiments.

A user interface with a keyboard 414 and mouse 416 can be used by a clinician to observe events 414 on the screen detected during an EMG monitoring session. In various aspects of the disclosure, the detected events can be used by the clinician concurrently with titration of the stimulus parameters, including pulse amplitude using the VNS stimulator and also selection of an active electrode or electrodes.

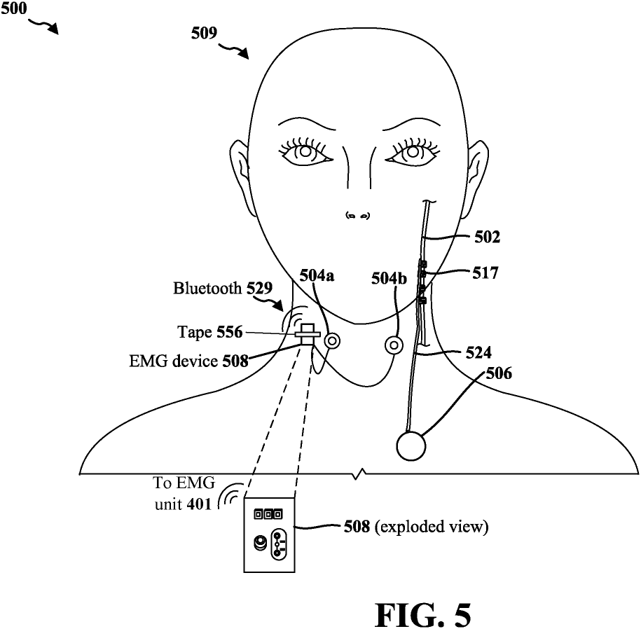

To this end, FIG. 5 shows an example diagram 500 of an external wireless EMG sensor system used by a clinician in some embodiments. A patient 509 is shown along with an exemplary front view of the patient’s larynx. It will be appreciated that the larynx is not exposed via surgery (i.e., it is underneath the skin surface), but the patient is awake and conscious and the larynx is being shown through the patient’s skin for clarity. In an embodiment, the patient 509 has a VNS stimulator 506 implanted under the skin in the chest area. As before, the VNS stimulator is coupled via lead wire 524 and electrode cuff 517 to vagus nerve 502. It will be appreciated that the structures in FIG. 5 are not necessarily drawn to scale, but are instead shown for clarity.

An EMG sensor may be placed on either side of the larynx. The EMG sensor includes a pair of surface electrodes 504a and 504b coupled to a small wireless transceiver 508. The wireless transceiver 508 may be affixed using tape 556 or some other adhesive means or otherwise externally affixed to a region on the skin adjacent the larynx, such as a throat area of the patient 509. An exploded view of the transceiver 508 is shown. Coupled to transceiver 508 are the two surface electrodes 504a and 504b attached over the skin to each side of the larynx. In other configurations, a single electrode or multiple electrode may be used across the larynx or different areas of the throat as determined in the discretion of the clinician.

The procedure as described herein is anticipated to be used following the initial system implantation and the periodic post implantation periods (conducted annually or otherwise). Measurements from the EMG sensor 508 can be transmitted wirelessly, e.g., via Bluetooth Low Energy to minimize interference, to the EMG unit 401 to enable the clinician to observe the EMG results via monitor 414.

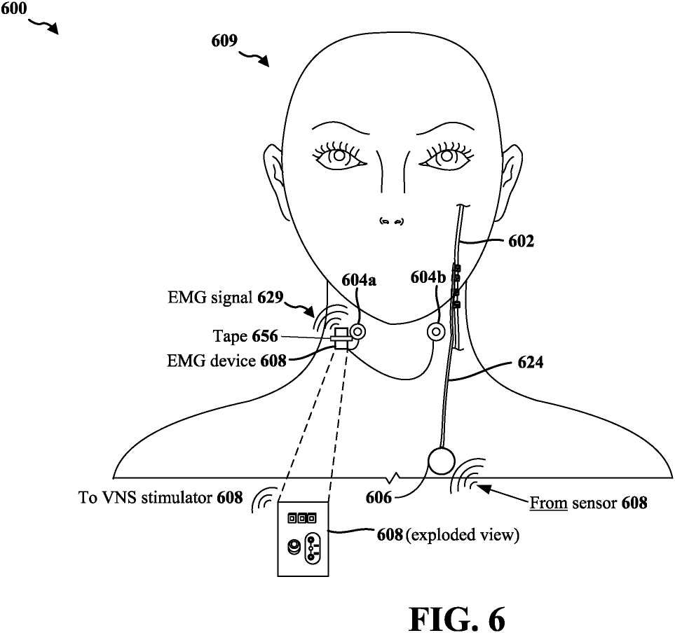

FIG. 6 shows another example of the wireless EMG sensor system for sending data directly to the VNS stimulator in some embodiments. As before, a clinician may configure a patient 609 with an electrode 604a and 604b on each side of the throat. The clinician may use tape 656 or other mechanism to affix compact EMG device on the neck area. VNS stimulator 606 may include a wireless receiver as shown in the text “from sensor 608” in FIG. 6. Lead wire 624 is connected to the vagus nerve 602. This embodiment includes further automation. Instead of transmitting the signal to the EMG unit 401 as in the embodiment described with reference to FIG. 5, the EMG signal 629 of FIG. 6 can be additionally or alternatively transmitted by the EMG device 608 to the wireless (or wired) receiver 216 on the VNS stimulator 204 of FIG. 2 (or in this example, to the VNS stimulator 606). The controller 220 can use its processing power to identify relevant events in the readings performed by the EMG device 608, and can titrate the value of the one or more stimulus parameters, e.g., the stimulus amplitude accordingly. In some embodiments, the signal 608 can also be provided to the EMG unit 401 where the readings are also made available to the clinician as well as the VNS stimulator 606. In this manner, subject to any overriding signal from the clinician, the controller 220 can automatically titrate the stimulus parameter, for example, the stimulus amplitude upward until the EMG readings are construed to imply otherwise. Advantageously, computations regarding the amount of the titration and the side effects or other events detected by the EMG may be substantially faster than solely through conventional clinician action. With the initial need to select sets of active electrodes when there is an array of (multiple) electrodes, as well as the need for initial and subsequent titrations with a large number of combinations of stimulus parameters, the use of objective EMG readings facilitates the titration process from the perspective of automaticity, time efficiency, and accuracy.

In various embodiments, additional external sensors can be used concurrently with the EMG sensor to identify other events relevant to acceptable amplitude levels. Certain such embodiments are described further below. Referring back to FIG. 5, when the EMG sensor as described above is affixed externally on the patient to the larynx region of the throat, the EMG sensor (e.g., transceiver 508 and electrodes 504a-b) may be used to detect physical events-in this case, for example, efferent stimulation on the vagus nerve 502-from the patient. A larynx muscle contraction may also be detected, or other events indicative of side effects or unwanted phenomena. It may be beneficial to avoid having any of the vagal stimulation pulses reach the laryngeal branch of the vagus nerve 502.

The clinician can select the position of the appropriate surface electrodes 504a-b (also known as the cathodes) to avoid this type of interference. The clinician or the controller 220, in the case of full automation, can use the readings from the EMG sensor to slowly titrate up or, as needed, step down the pulse amplitude of the VNS stimulator 506 over time. For example, the clinician can use the monitor 414 to make readings based on the events detected from the EMG sensor. As another example, once efferent stimulation on the vagus nerve 502 is detected as indicated by EMG readings, or other bodily events are detected relevant to identifying an acceptable stimulation amplitude as described below, the clinician or controller may stop titrating the amplitude, via the controller or other conventional methods used for titrating the stimulus amplitude. In some embodiments, for certain side effects identified by the EMG sensor (e.g., contraction of the larynx), the controller 220 or clinician may determine that a downward titration is necessary.

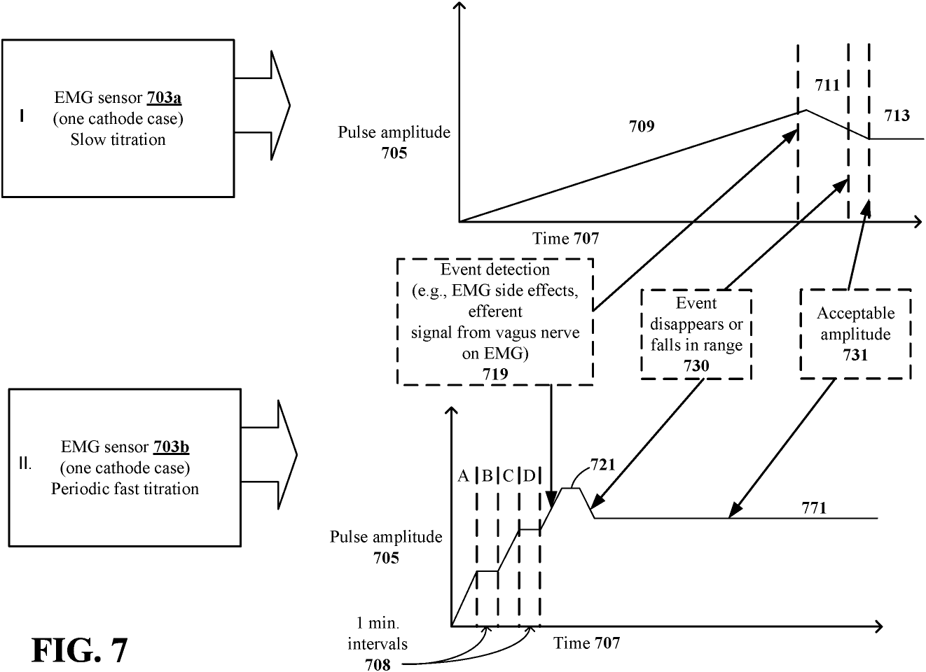

In another embodiment, in lieu of slowly titrating up the dosage, the clinician may elect to quickly increase the pulse amplitude. FIG. 7 is an example of an embodiment of example titration processes I and II using an EMG sensor to detect physical events from the patient using EMG sensors 703a and 703b, respectively. In situation I, titration is slowly increased as described above. The readings from the EMG sensor may be received wirelessly at the EMG unit 401 as above. The EMG sensor 703a is placed on the larynx, such as by using one surface electrode on each side of the throat.

FIG. 7 shows a graph corresponding to situation I. The graph is not drawn to scale. The vertical axis represents a pulse amplitude 705 of the VNS stimulator. The horizontal axis represents time 707. Region 709 of the graph shows the clinician using conventional measures to titrate upward the stimulation current amplitude to the vagus nerve. At the leftmost dashed vertical line as indicated by the arrow leading from box 719, the EMG sensors may detect at least one physical event from the patient, which may include, among other events as described herein or otherwise, an efferent stimulation on the vagus nerve. As a result, the clinician (or the controller, in fully automated systems such as in FIG. 6) may proceed to stop the upward titration or, in this embodiment, may titrate the amplitude of the electrical VMS pulses downward, as in region 711. After a short delay while the message is received and processed, the amplitude reverses and ramps downward in time in region 711. The amplitude of the pulses proceeds to slowly reduce as it is titrated downward until the effect disappears. Advantageously, in some embodiments, this downward titration may require only a single downward step. Other embodiments may require more downward titration until the detected side effect(s) disappears.

At the vertical dash corresponding to the arrow from box 730, the readings at the EMG unit show that the physical event may change significantly or disappear. At that point, the clinician or controller 220 stops the downward titration. In this example, it is assumed in region 713 of the graph that the electrical stimulus pulses have reached an acceptable amplitude (box 731 and corresponding arrow).

In some embodiments depending on the types of events that the EMG sensor and one or more other sensors are measuring, the upward/downward titration cycles may continue to occur until the acceptable amplitude is achieved. Nevertheless, the graph corresponding to FIG. 1 is representative of the slow titration trend according to an embodiment. The total time for the acceptable amplitude to be achieved may change depending on medical considerations, the patient anatomy, the objectives of the treatment, and other factors.

In situation 2, an EMG sensor 703b may be coupled externally to the patient’s throat, as shown previously. This situation identifies the embodiment in which the amplitude of electrical stimulus pulses to the vagus nerve is quickly increased (such as every minute, for example) as the EMG sensor data is monitored to determine if an efferent signal is generated at the vagus nerve. Thus, a unique efferent stimulation current amplitude can be determined for the electrode/cathode.

The lower right of FIG. 7 shows an exemplary graph corresponding to this embodiment (not drawn to scale). As before, the vertical axis represents the pulse stimulus amplitude 705 and the horizontal axis represents time 707. In this example, the pulse amplitude to the vagus nerve may be increased rapidly at region A. The titration may stop at the first vertical dashed line. At region B of the graph, an interval of time may occur (e.g., one minute or the like) prior to restarting the titration. At C, the titration of the amplitude recommences rapidly upward for a short time, and then stops again for region D, where another time interval may be inserted to allow the body to adjust to the increase.

After the region D, fast upward titration may commence. In this example, an event is detected at the EMG sensor 703b as shown by the arrow from box 719, prompting the clinician or controller 220 to stop the titration. The amplitude thus peaks at 721 and in this embodiment, the clinician (or the controller 220 directly) titrates the amplitude downward. The events detected from the patient using EMG sensor 703b may change or disappear as shown by the arrow from box 730, again prompting the clinician or controller to stop the downward titration. An acceptable amplitude may be reached thereafter as shown by the horizontal line 771 on the graph and as shown from the arrow from box 731.

In contrast to prior approaches, the combination of the EMG sensor with the stimulator may allow for a prompt determination of acceptable stimulus parameters, e.g., the acceptable stimulation amplitude of the vagus nerve. In addition to the single electrode case, the above example can also extend to the situation where a quick determination can be made for selecting each active electrode or electrode pair in a multi-electrode stimulation cuff or lead, as described below.

In one embodiment, the stimulation amplitude from the VNS stimulator 204 may be increased for a particular active electrode as long as the EMG sensor does not detect an event like a muscle contraction event coincident with the stimulus pulses. Thereafter, another electrode or cathode, or pair of cathodes, may be titrated upward in a manner similar to the embodiments described above. A unique efferent stimulation threshold may be determined for every anode/cathode pair. The controller 220 can thereupon save these thresholds to the memory 214 (FIG. 2).

It is expected that when a pair of cathodes are used to deliver stimulation concurrently, the acceptable stimulation amplitude for the pair of cathodes will be lower than when each electrode in the pair is activated alone. This is the reason why pairs of electrodes functioning concurrently as cathodes may each be treated as a unique electrode pair during the titration process. Thus, pairs of cathodes may have their own maximum stimulation amplitude when driven as a pair. In some embodiments, two or more cathodes may be used with only one anode as a current return path.

Other external sensors can be used, concurrently or during different periods. In another aspect of the disclosure, during the titration of the stimulation amplitude and the cathode selection after the initial fitting, an EKG may be monitored to detect possible ictal tachycardia events as a surrogate for seizure detection.

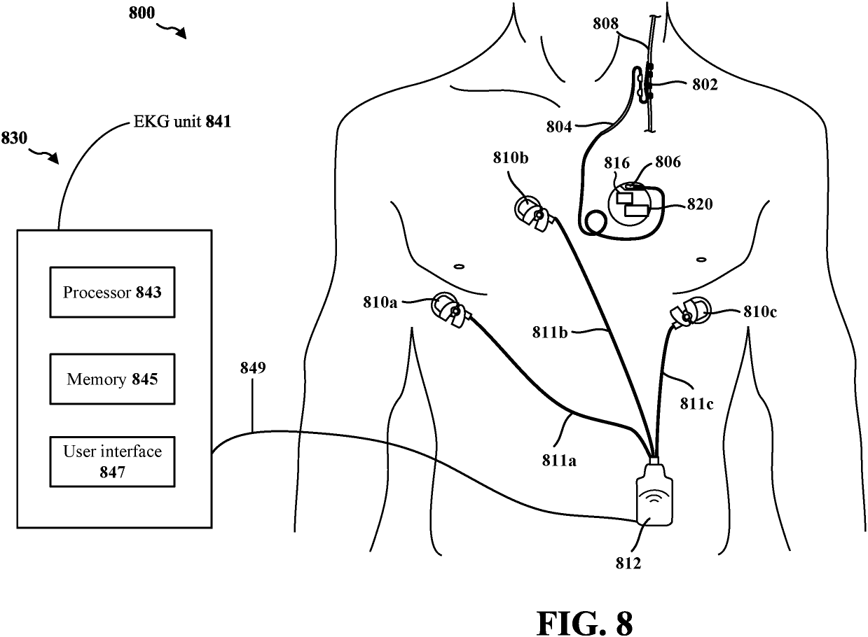

FIG. 8 is an illustration 800 of an embodiment of a patient equipped with an external EKG sensor 830 and an implanted VNS stimulator 806. Wires may run from external compact device 812 to an EKG monitor unit 841 analogous to the EMG unit 401. In the illustrated embodiment, the compact device includes a processor and an interface for coupling to EKG lead wires 811a, 811b, and 811c. The EKG lead wires may terminate in respective lead wire terminals for attaching to corresponding lead wires 811a, 811b and 811c, the latter of which are taped or otherwise affixed on selected regions of the patient. In various embodiments, different numbers of lead wires and electrodes may be used (e.g., five electrodes, etc.). The compact device 812 may include one or more wires 849 directly attached to the EKG unit 841 for sending data from the surface electrodes 810a-c to the EKG unit 841. In the embodiment shown, EKG unit 841 may be a larger dedicated unit for receiving and measuring detected physical events at electrodes 810a-c. The EKG unit 841 may include a processor for running instructions that the controller 841 stores in the memory 845. The memory 845 may also store the detected events for evaluation by the processor 843 or by another device. In addition, a user interface 847, which may include a touch screen, keyboard, mouse, and the like, may be part of EKG unit 841.

In other embodiments, the wire 849 may be absent and the compact device 812 may include a wireless transmitter for sending wireless transmissions to an EKG unit pursuant to any number of wireless protocols.

VNS stimulator 806 may be implanted in the patient via lead wire 804 to the vagus nerve 808. The signals may be read by the controller 820 for use in titrating the stimulus parameters, including stimulus amplitude for one or more electrode pairs 802. Alternatively, or in addition, the compact device 812 may send the signals from the lead wires 811a-c to wireless transceiver 816 on VNS stimulator 806 for providing the signals directly to the controller 820 on VNS stimulator 806.

More generally, an EKG is an external, sensor-based system used for recording the electrical signals in the heart. EKGs may be used to detect abnormal heart rhythms (arrhythmias), evidence of blocked arteries, pacemaker analyses and other events relevant to functioning of the heart.

In another aspect of the disclosure, physical events from an EKG are measured and provided to the clinician and/or a controller via user interface 847 or via controller 820 of the VNS stimulator 806. The clinician may use a monitor as part of the user interface 847 to view the signals from the electrodes. During titration of stimulus amplitude for the vagus nerve 808 and selection of active electrodes-cathodes following the initial fitting, the EKG may be monitored to detect ictal tachycardia events as a surrogate for seizure detection. In various embodiments, the EKG sensor may be used concurrently with other sensors, or on its own.

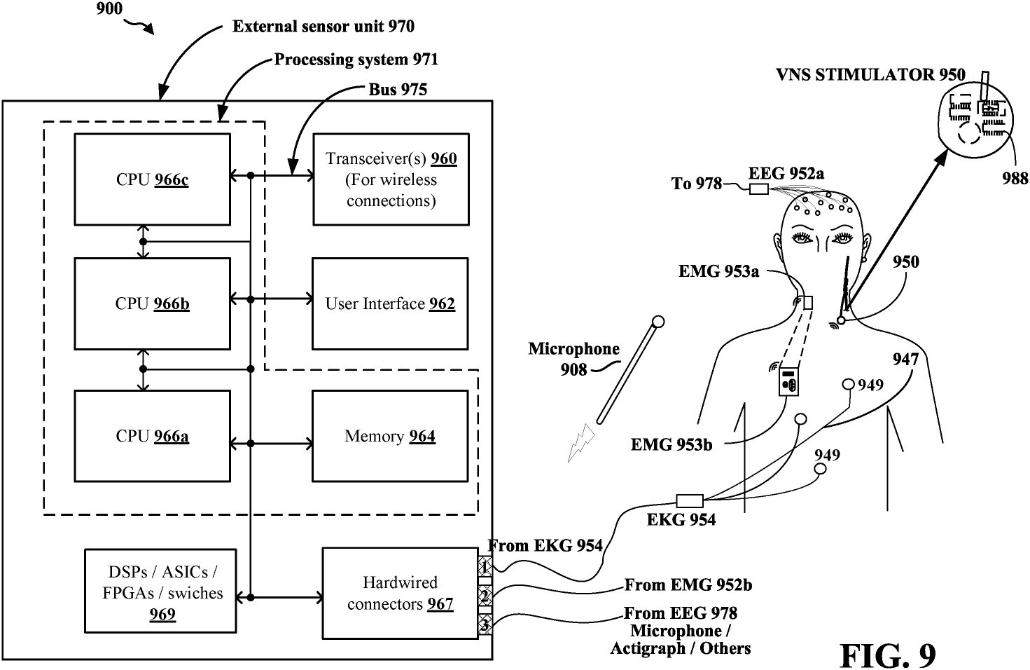

In other embodiments, a microphone (FIG. 9) may be used to monitor heart rate and detect ictal tachycardia events. In still other embodiments, the stimulus amplitude may either be increased from a minimum value, or decreased from the maximum value previously determined using the larynx EMG sensor as described in embodiments above. In yet other embodiments, a wireless microphone with an onboard inertial measurement unit (IMU), or a Micro-Electro Mechanical Systems (MEMS) microphone may be used to detect various physical events including cough, which is one of the common side effects of VNS stimulation. Advantageously, certain external sensors such as these can be used in an outpatient setting, including at the patient’s home.

These embodiments rely on the fact that an increase in heart rhythm is common during a seizure. One type of epileptic seizure is known as ictal tachycardia, in which the subject’s heart rate increase of more than ten (10) beats per minute of above the baseline. Epileptic seizures can lead to changes in autonomic function affecting the nervous systems. Changes in cardiac signals are potential biomarkers that may provide an extra-cerebral indicator of seizure onset in some patients. As a result, EKG sensors can assist the controller 220/820 in detecting cardiac events, including their number and magnitude, that may be associated with an upcoming seizure. Accelerations of cardiac events can be measured, thereby allowing the early detection of arrythmias (such as ventricular fibrillation) that may cause death. It is generally reported that significant heart rate changes are associated with a large number of patients that experience epilepsy. The EKG sensor and/or a microphone or stethoscope can be used to detect these changes in heart rate, including ictal tachycardia events.

In various embodiments, each change in stimulus amplitude made based on the number of ictal tachycardia events or other heat rate phenomena can be monitored for a relatively long duration, for example, about one week. The controller 820 (FIG. 2) may store these events in memory. The clinician and/or controller can compare the average number of ictal tachycardia events, e.g., over the week or other duration of time. If the change in the number of ictal tachycardia events is significant, such as above some threshold, the controller 820 or clinician may increase the pulse amplitude again to attempt to increase the efficacy of the VNS stimulator. This comparison may be repeated for two or more periods of time until no further reduction in ictal tachycardias is observed.

In some embodiments, the above information can be maintained and processed using a processing system external to the patient. Thereafter, relevant information about the detected EKG events can be downloaded to the controller. In some embodiments where the data is processed externally or by a clinician, an external processing system may send an instruction to the VNS stimulator to titrate the stimulus parameters, e.g., stimulus or amplitude. In still other aspects, while monitoring for ictal tachycardias, the EKG sensor can also monitor, and a processor can maintain a count of, the total number of bradycardias. A bradycardia is a slower than normal heart rate, such as less than sixty beats per minute. A bradycardia can stop the brain or other vital organs from receiving enough oxygen, which can result in various side effects and potentially dangerous symptoms. An increase in bradycardias can be an undesirable side effect of high stimulation. Accordingly, in various embodiments, where the EKG or other external sensor identifies some threshold number of bradycardias, the periodic increase in stimulus amplitude may be halted.

Other physical events can be relevant to an acceptable stimulus pulse amplitude. An ACTi graph is a type of accelerometer that may measure sleep parameters and motor events over the course of days or weeks. These events may be relevant to titrating a stimulus amplitude. Prone position events such as acute respiratory distress may be measured by a ventilator. Still other heart rate events may also be measured, such as low heart rate events. Heart Rate Variability (HRV), which can also be measured by an EKG, can be measured so that the efficiency of VNS therapy on patients with epilepsy who have bradycardia or normal heart rate can be compared to patients who have ictal tachycardia.

Another aspect of the disclosure involves patient feedback through a user interface coupled to or included within an electronic device, such as a specialized medical device or a general purpose computer (PC, mobile phone, laptop, etc.). Some aspect of stimulation may be increased autonomously. This stimulation may occur in some instances without the need for patient notification. In other instances, a mechanism for the patient to provide feedback may be made available through the patient’s controller 220 (FIG. 2), which may include an application on a PC or mobile phone, or other device. In various embodiments, this mechanism may include a button, switch or other selection means included on the patient controller. The patient may press the button or select the switch if the patient or clinician (caregiver, physician, and the like) notices an event from the patient. An event may include side effects. Common such side effects may include a voice change, a sore throat, heart palpitations, difficulty swallowing, paresthesia, insomnia, shortness of breath, and the like. In some embodiments, the patient controller may include queries for the client to answer (such as in the computer application on the patient controller) to enable the patient controller to determine the nature of the patient’s problem. In other embodiments, the application may present a periodic survey to prompt the patient/caregiver to assess whether side effects due to the VNS stimulation have occurred. The feedback provided by the patient as a result of the survey may indicate that the patient has experienced no side effects and is comfortable. This information may also be used to prompt the next change in titration of the pulse amplitude.

The ability to view long term data or trends of the patient’s tolerance to each adjustment to the amplitude can help determine whether one or more side effects may be decreasing over time. Some configurations may involve a wireless microphone (908) with an onboard inertial measurement unit (IMU) or a Micro-Electro Mechanical Systems (MEMS) microphone, may be used to detect the patient’s cough, which is one of the common side effects of VNS stimulation, and record the data in the patient controller.

FIG. 9 is an example block diagram 900 of a patient outfitted with different external sensors and an example sensor unit including a processing system according to an embodiment. FIG. 9 includes an external sensor unit 970 for assessing one or more of EKG, EEG, EMG sensor data, and other sensor data such as auditory and inertial external sensor data. It should be understood that the unit 970 in practice may instead be multiple units that specialize in processing data for different respective sensors. Thus, external sensor data may in practice include an EEG unit, an EKG unit, and EMG unit, and so on. In various embodiments, unit 970 may include a general purpose processing system such as a personal computer, a server, or the like. Other embodiments may include multiple units corresponding to multiple sensors along with a central processing system housed in one of the units (or another unit) for consolidating and analyzing the data.

A patient may be adorned with EEG sensors 952a. The sensors may include lead wires that terminate at a compact EEG device 952a. The compact EEG device 952a may be connected via hardwire as shown in hardwired connector 967 at port 3. In some embodiments, the compact EEG device 952a may instead transmit its data to the unit 970 using a suitable wireless technology. In addition, in some embodiments, either unit 970 or compact EEG device 952a may be configured to transmit data to the VNS pulse stimulator 950 for use by controller 988, wirelessly or otherwise.

The patient may also wear an EMG unit 953a (exploded view in 953b) along with EMG electrodes (not shown) to enable a clinician to perform EMG tests. The EMG unit 953b may be hardwired to hardwired connector 967 at port 2 to provide the external sensor data to external sensor unit 970 (e.g., an EMG unit). In various embodiments, the EMG unit 953a may instead transmit the sensor data using Bluetooth Low Energy, or another wireless technology.

A microphone 908 and other auditory sensors may be used to determine cardiac events. The information may be provided to a user interface 962 (e.g., where unit 970 is a personal computer or specialized external unit). In some embodiments, microphone 908 may include a wireless connection to transmit information wirelessly to the unit 970 or the VNS stimulator 950.

Referring still to FIG. 9, an expanded view of VNS stimulator 950 is shown. Controller 988 on the VNS stimulator 950 may in some embodiments receive data or instructions, wirelessly to external sensors and/or unit 970 or through a temporary wired connection, that connects the implanted stimulator to external sensors and/or unit 970. VNS stimulator 950 can use these data or instructions to titrate the amplitude of the electrodes on the pulse generator in some embodiments.

The unit 970 in FIG. 9 may further include a transceiver 960 to effect one or more wireless connections, whether to or from the sensors as described above, or to or from controller 988. Where unit 970 includes a plurality of units specific to different sensors, transceiver 960 may be used to obtain data regarding physical events to titrate the stimulus parameters, e.g., stimulus amplitude. In other embodiments, the stimulus amplitude may be titrated using the external sensor unit 970.

The patient may also, concurrently or at a different time, be outfitted with EKG electrodes 949. EKG electrodes may provide data regarding heart-related events through lead wires 947 to a compact EKG device 954. In some embodiments, EKG device 954 may include wires to the external sensor unit 970 (e.g., an EKG unit) at port 1, as shown in FIG. 9. EKG device 954 in some embodiments may be configured as a wireless device, providing the EKG sensor data to the external sensor unit 970 using transceiver 960. Further, in various embodiments, either EKG device 954 or external sensor unit 970 may transmit EKG sensor data or instructions relating thereto to the VNS stimulator for use by controller 988.

In various embodiments discussed above with reference to FIGS. 4 and 5, the compact device affixed to the patient’s neck may automatically transmit the detected information to a unit, such as transceiver 220 on the VNS stimulator 218. In other embodiments, the transceiver (220) may also send the information to a unit like unit 970 to enable the clinician to review the results. Unit 970 may also include a bus system 975 to connect all the components together. A memory 964 may be used to store data corresponding to the outputs of one or more sensors. The memory may include one or more hard drives (solid state or magnetic), DRAM, SRAM, programmable memory such as PROMs, EPROMs, EEPROMs, flash memory, and/or other volatile and nonvolatile means of storage. In addition, various types of hardware components 969 (e.g., DSPs, ASICs, FPGAs, switches and other devices may be used, and in some cases, included at least in part as a portion of the processing system.

Processing system 971 may include one or more CPUs 966a-c and memory 964. The processing system in some embodiments may be configured to evaluate the received sensor data including (i) specialized EMG data if the external unit 970 is an EMG sensor, for example, or (ii) multiple physical events from multiple sensors, if the external unit 970 is configured to include a sophisticated processing system with code to recognize and evaluate different types of sensor data.

In various embodiments, the processing system 971 can apply weights and significances to these events and can determine, using the consolidated sensor data, an appropriate titration schedule. The processing system 971 is shown to include CPUs 966a- c, but in other embodiments the processing system 971 may perform one or more functions, at least in part, using dedicated hardware 969. In various embodiments, the processing system uses the hardwired connectors 967 or the transceiver to transmit data to, or receive data from, one or more sensor as well as the VNS stimulator 950. Using multiple external sensors can provide significant advantages in identifying an optimal set of pulse amplitudes for a multi cathode cuff, for example. The combination of different such measurements may in some cases be reinforcing, and the probability of success in titrating the stimulus parameters and, particularly, stimulus amplitude based on a combination of physical events may provide a maximally acceptable amplitude at each electrode/cathode for reducing seizures or depression, etc.

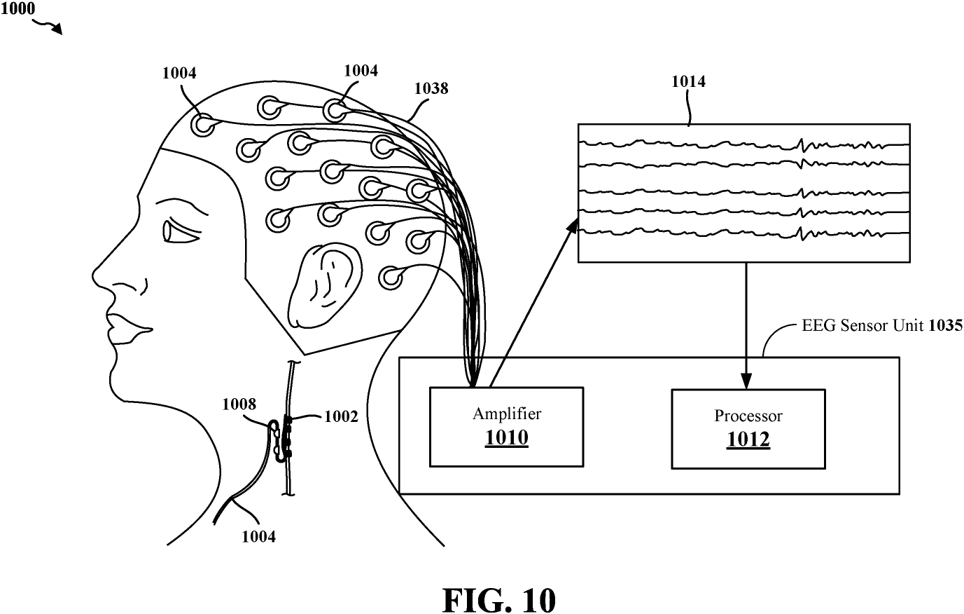

FIG. 10 is a conceptual diagram of an EEG sensor affixed to a patient’s head and a device for interfacing with a processing system. An EEG, or electroencephalogram, measures electrical activity, including abnormal activity, in the brain. The clinician may place a flexible cap or connected assembly of small electrodes 1004 (here, conducting discs) on the scalp. The signals from the brain flow through the lead wires 1038 to an EEG sensor unit 1035 (similar to the EKG and EMG units). The sensor unit 1035 includes amplifier 1010. Because the electrical signals from the brain are very small, the amplifier 1010 can be used to boost the signal strength to a level that processor 1012 can utilize. The EEG sensor unit 1035 can include additional components, including memory, dedicated hardware for performing specific tasks, and a user interface panel. The user interface may include screen 1014 in which the electrical activity can be viewed.

In measuring brain activity, the EEG can also recognize improvements and therapeutic effects as brain activity stabilizes (e.g., as a result of the pulses generated by VNS stimulator 204). For example, at the outset of titration therapy, the EEG can make measurements as a baseline, and in subsequent sessions over various intervals, the EEG sensor unit 1035 can compare measurements with the baseline measurements. In some embodiments, the EEG sensor unit may include a transceiver or transmitter for sending information to controller 220 on the VNS stimulator 204 (FIG. 2). Also, in various embodiments, the EEG sensor unit 1035 may be coupled to a separate compact device (similar to compact device 812 in FIG. 8 as in the EKG embodiment), to mediate the flow of signals from the brain and to the EKG sensor unit 1035.

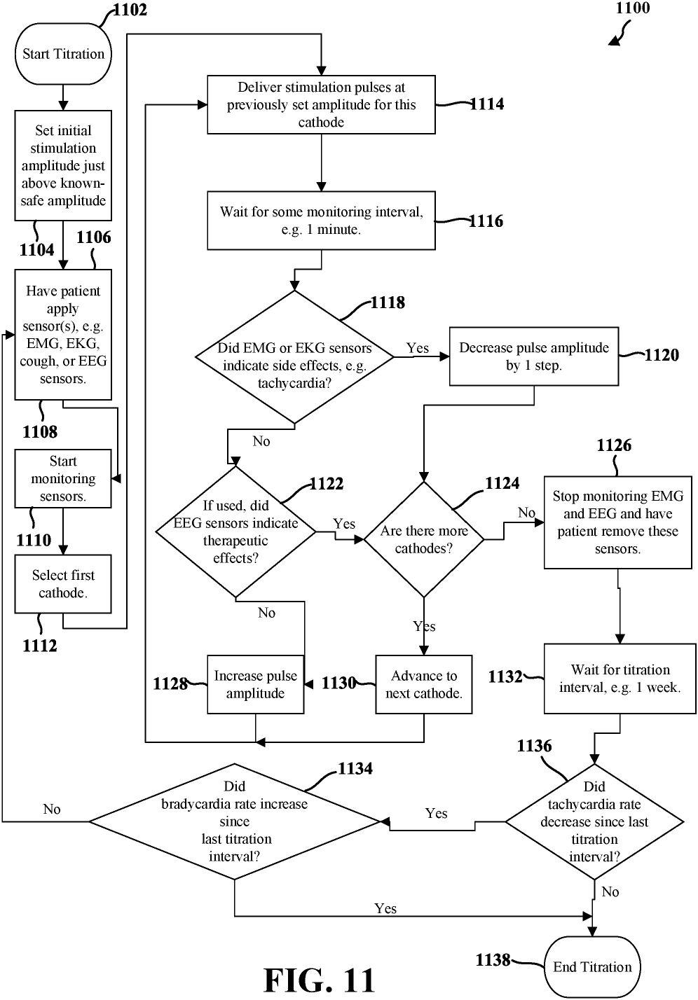

FIG. 11 is a conceptual flow diagram of a process for performing automated VNS titration according to an embodiment. The method for performing the steps in FIG. 11 may be performed by one or more of the VNS stimulator and associated (see FIGS. 1-3 and accompanying text); external EMG sensors (FIGS. 4-7), EKG sensors (FIGS. 8 and 9), EEG sensors (FIGS. 9 and 10); microphone 908 (FIG. 9), a separate patient controller and other external sensors not specifically identified and used in connection with automated VNS titration. It should be understood that the steps described in FIG. 11 are exemplary in nature, and other embodiments are possible. The steps may be monitored or controlled by a clinician and/or controller 220, or some combination thereof.

It is assumed that the patient is present and one or more external sensors, such as EMG, EKG or EEG sensors, are used for making measurements as described above. In various embodiments, one external sensor may be used at a time, such that different sensors may be used and the data may be separately gathered. In other embodiments, certain external sensors can be simultaneously used.

At 1102, titration is started. This may occur, for example, at the beginning of the implantation after surgery and a suitably determined recovery period. At 1104, the clinician or controller 220 (FIG. 2) may set an initial stimulus parameters, e.g., stimulus amplitude of electrical pulses that may be either below or at a known safe, but expected therapeutic stimulus amplitude. The frequency, pulse width, and in some embodiments the duty cycle have already been set to selected values. At 1106, with the VNS stimulator 204 now functional, a clinician may outfit a patient with one or more external sensors, such as an EMG, EKG or EEG sensor, or a microphone or other sensing device.

At 1108, monitoring of the sensors actively commences. The monitoring may occur by a clinician recording data or, as noted above in other embodiments, by the controller 220 receiving the sensor data wirelessly at the VNS stimulator 204. In still other embodiments, the data may be processed by a larger unit such as the EMG unit of FIG. 4, or the EKG/EEG/EMG unit 870 (FIG. 8). A server computer or personal computer may also be used for these purposes, with or without active clinician involvement. The data received by the sensors may be stored in memory or otherwise recorded.

In a multiple electrode scenario involving electrode pairs, a first cathode may be selected (1112). At 1114, the controller 220 may have the pulse generator 306 deliver stimulation pulses at the previously set stimulus amplitude for the selected cathode. A predetermined time may therefore be set (for example, one minute) during which the pulses are delivered. After waiting for the predetermined time (e.g., 1 minute, or in some cases, hours or days), at 1118, the data from the external sensors (e.g., one or both of the EMG or the EKG sensors) are analyzed to determine whether physical events from the patient (e.g., side effects such as tachycardia) are present. If side effects are present, then at 1120, the clinician or the controller 220 may decrease the amplitude by one step (e.g., a fixed amount).

Conversely, if no relevant physical events are observed, then in the case where EEG external sensors are used, the brain activity from the EEG sensors are analyzed to determine whether therapeutic effects are indicated. Therapeutic effects may include physical events that demonstrate that abnormal brain activity is stabilized. If not, then the pulse amplitude may be incrementally increased (1128). The cycle may start again at 1114 where the increased pulse amplitude is delivered for the cathode at issue (in a multi-electrode cuff), and the above considerations are again taken into account. If therapeutic effects are in fact demonstrably present by virtue of EEG sensor use, then the pulse amplitude can be stored in memory as an acceptable stimulus pulse amplitude.

At 1124, the clinician or controller 220 determines whether additional cathodes are present at the VNS cuff. If so, then the controller 220 or clinician using external equipment (or controlling the VNS pulse stimulator) advances to the next cathode and again, the process repeats itself at 1114, wherein the controller 220 causes the pulse generator 206 to deliver stimulus pulses to the vagus nerve at the previously set amplitude for the cathode in question.

Referring back to 1118 and the results of the EMG or EKG sensors, if relevant physical events are detected on the cathode in question (or on the electrode or electrode pair in a single electrode configuration), then as noted above, the pulse amplitude may be decreased (or decreased again, if the same cathode has previously been through this portion of the flow diagram) at 1120 by one step. Control may flow to 1124, in which it is determine whether additional cathodes are present. If so, as before, the controller 220 advances to the next cathode until all measurements are made and all upward or downward steps are taken on each of the cathodes.

When all cathodes have been set at 1124, then at 1126, the clinician stops the monitoring of the EMG and EEG sensors and assists the patient in removing the sensors. Here, based on the various physical events identified by one or more sensors, the acceptable stimulus amplitudes of the cathodes are set. In one implementation at 1132, an amount of time allotted, such as one week (although the amount of time may vary widely depending on the circumstances and the overall clinical situation, and also the disposition of the patient), is set as a titration interval, and the patient and clinician wait a week.

After the identified time has passed, the patient may be equipped with external sensors, or the clinician may use a microphone or other auditory sensor. At 1136, the clinician determines whether physical events such as the tachycardia rate decrease since the last titration interval. If not, the professional(s) overseeing the procedure may consider ending the stimulation treatments, and titration may be ended at 1138. If so, the clinician may also review the sensor data to determine whether the bradycardia rate increased since the last titration interval. If so, then again the professionals may elect to end titration (1138). If the bradycardia rate did not increase since the last interval, then at 1106 the clinician can have the patient apply various external sensors, including EMG, EKG, cough or EEG sensors. The monitoring session can restart at 1110, and the sensor data can be used as described above and in FIG. 11 to determine the acceptable amplitudes for the cathode or multi-cathode system.

The titration to find an acceptable stimulus parameters, and particularly stimulus amplitude can be advantageously increased and is more precise than existing methods using the principles of the present disclosure because among other benefits, the external sensors can accurately produce data regarding physical events from the patient and an acceptable pulse amplitude can be rapidly found.

In certain embodiments, the controller can accumulate data from both internal and external sensors and remotely send the data to the clinician for clinician’s review. The clinician can then remotely program the next titration step based on the current sensor data and patient’s history which could avert some undesirable side effects and expedite the titration by providing personalized care without performing in-clinic follow-up. The decisions at step 1118, 1128, 1130, 1134 and 1136 in FIG. 11 are being performed by clinician and next titration steps are programmed remotely.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub- combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub- combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above- described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators-such as “first,” “second,” “third,” etc.-for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

What is claimed is:

1. A system for vagus nerve stimulation (VNS), comprising:
a VNS stimulator implanted in a patient and configured to transmit periodic or episodic electrical stimulation pulses to a vagus nerve of the patient; and
a sensor configured to detect from the patient a biological signal,
wherein the VNS stimulator comprises a controller configured to automatically titrate at least one of a VNS pulse stimulus parameter, among stimulus pulsewidth, stimulus amplitude, stimulus frequency and duty cycle, based at least in part on the biological signal until an acceptable stimulation result is achieved.

2. The system of claim 1, wherein the sensor comprises an electroencephalogram (EEG) sensor, the EEG sensor being positioned on a patient’s scalp or behind a patient’s ear.

3. The system of claim 1, wherein the biological signal comprises a predetermined electrical activity in a patient’s brain.

4. The system of claim 1, wherein the sensor comprises an electrocardiogram (EKG) sensor.

5. The system of claim 1, wherein the biological signal comprises one or both of an intensity or frequency of ictal tachycardia or bradycardia events.

6. The system of claim 1, wherein the sensor comprises an electromyography (EMG) sensor.

7. The system of claim 6, wherein:
the EMG sensor is configured to be positioned on or proximate to a larynx of the patient and to detect, as the event, stimulation information of the laryngeal branch of the vagus nerve; and
the controller is configured to titrate up at least one of the VNS pulse stimulus parameters, which includes pulse width, pulse frequency, pulse amplitude, frequency and duty cyle, over a period of time based on stimulation information including side effects of the patient relating to a detected stimulation of the laryngeal nerve.

8. The system of claim 1, wherein the controller is configured to automatedly use the biological signal to determine a unique stimulation threshold for at least one electrode of the VNS stimulator when the VNS stimulator is configured using either a single electrode, or configured using a multi-electrode stimulation cuff or lead.

9. The system of claim 1, wherein the controller is configured to determine a VNS stimulation threshold by increasing the VNS pulse amplitude at one or more intervals over time until the sensor detects a biological signal from the patient relevant to an acceptable amplitude of the stimulation pulses.

10. The system of claim 1, wherein the sensor comprises a microphone, and the biological signal comprises a heart rate or an ictal tachycardia event.

11. The system of claim 1, wherein the controller is configured to determine an efficacy of the VNS stimulator based on measurements from an EKG sensor or an EEG sensor,
wherein the measurements from the EKG sensor include any one or more of heart rate variability (HRV) measurements, bradycardia events, or tachycardia and fibrillation events.

12. The system of claim 11, wherein:
the measurements from the EKG sensor comprise one or more of a number of ictal tachycardia events, a number of bradycardia events, or a Heart Rate Variability (HRV); or the measurements from the EEG sensor comprise indications of a seizure event.

13. The system of claim 1, wherein the controller is configured to automatically effect an optimal titration of the VNS stimulation amplitude such that seizure events are reduced with minimal stimulator use as determined based at least in part on the event.

14. A method for vagus nerve stimulation (VNS), comprising:
transmitting periodic electrical stimulation pulses from a VNS stimulator implanted in a patient to a vagus nerve;
receiving, from a sensor external to the patient, data comprising a physical biological signal from the patient and relevant to an acceptable stimulation of the vagus nerve; and
titrating an amplitude of the pulses upward based at least in part on the data.

15. The method of claim 14, wherein the received data is included in a wireless signal.

16. A device for automatically titrating a vagus nerve stimulation (VNS), comprising:
a VNS stimulator implanted in a patient and configured to transmit electrical stimulation pulses to a vagus nerve of the patient, the VNS stimulator comprising a controller configured to:
receive data from an external sensor configured to detect from the patient a biological signal relevant to an acceptable amplitude of the stimulation pulses; and
titrate an amplitude of the stimulation pulses based in part on the received data.

17. The device of claim 16, wherein the sensor comprises an electroencephalogram (EEG) sensor, the EEG sensor being positioned on a patient’s scalp or behind a patient’s ear.

18. The device of claim 16, wherein the event comprises electrical activity in a patient’s brain.

19. The device of claim 16, wherein the sensor comprises an electrocardiogram (EKG) sensor.

20. The device of claim 16, wherein the event comprises one or both of an intensity or frequency of ictal tachycardia or bradycardia events.

21. The device of claim 16, wherein the sensor comprises an electromyography (EMG) sensor.

22. A device for automatically titrating a vagus nerve stimulation (VNS), comprising:
a VNS stimulator implanted in a patient and configured to transmit electrical stimulation
pulses to a vagus nerve of the patient, the VNS stimulator comprising a controller configured to:
receive data from an external sensor configured to detect from the patient a physical event relevant to one or more stimulus parameters of the stimulation pulses; and
titrate at least one or more stimulus parameters of the stimulation pulses to find one or more optimal parameters based in part on the received data.

23. A device for automatically titrating a vagus nerve stimulation (VNS), comprising:
a VNS stimulator implanted in a patient and configured to transmit electrical stimulation pulses to a vagus nerve of the patient;
a cuff arranged on the vagus nerve and coupled to the VNS stimulator via a lead wire;
electrodes coupled to the cuff, the electrodes contacting the vagus nerve at different positions; and
a controller coupled to the VNS stimulator and configured to:
receive data from an external sensor configured to detect from the patient a biological signal relevant to one or more acceptable stimulus parameters of the stimulation pulses from at least one of the electrodes;
titrate one or more optimal parameters using different electrodes to find one or more optimal parameters and one or more optimal electrodes to activate as stimulating electrodes based in part on the received data.
